(19) United States
(12) Patent Application Publication
(10) Pub. No.: US 2011/0118733 A1
(43) Pub. Date: May 19, 2011

(54) ELECTROSURGICAL PENCIL

(76) Inventor: KUO-LIANG LIU, Taipei (TW)

(21) Appl. No.: 12/619,704

(22) Filed: Nov. 17, 2009

(51) Int. Cl. A61B 18/14

(52) U.S. Cl. 606/42

(57) ABSTRACT

An electrosurgical pencil comprises a housing being an elongated shank member, and including a conical head end with an opening fixed on a front end thereof, and including an upper housing part and a lower housing part connecting with each other, wherein the upper housing part includes a first and a second holes proximate to the opening; a control panel disposed in an interior of the housing, and including a controlling member secured thereon in response to the first hole and the second hole, and including an engaging head mounted at a front end thereof in response to the opening of the housing to retain a blade; two buttons fixed in the first and the second holes respectively to correspond to the controlling member of the control panel so as to turn on and off the blade, thereby enabling to switch the blade easily and to obtain a preferred stability.

### BACKGROUND OF THE INVENTION

1. Field of the Invention

The present invention relates to an electrosurgical pencil, and more particularly to an apparatus used in surgery.

2. Description of the Prior Art

As shown in FIG. 5, a conventional electrosurgical pencil 90 is applied in cosmetic surgery, and its shape is like a pencil so as to be held by a user to facilitate the surgery. A controlling method of a controlling member 91 (turning on and off operation) of the electrosurgical pencil 90 is to provide two buttons disposed on a middle section of the electrosurgical pencil 90 to turn on or off the electrosurgical pencil 90, hence the user allows to switch a blade quickly.

However, such a conventional electrosurgical pencil can not be operated easily, because as desiring to switch an operation of the blade during the surgery, the user has to move a forefinger from a head end of the electrosurgical pencil to the middle section thereof, accordingly the electrosurgical pencil is swung to cause an incorrect surgery.

The present invention has arisen to mitigate and/or obviate the afore-described disadvantages.

### SUMMARY OF THE INVENTION

The primary object of the present invention is to provide an electrosurgical pencil which is capable of overcoming the shortcomings of the conventional electrosurgical pencil.

Another object of the present invention is to provide an electrosurgical pencil which is operated precisely, stably, and easily.

An electrosurgical pencil in accordance with a preferred embodiment of the present invention comprises:

a housing being an elongated shank member, and including a conical head end with an opening fixed on a front end thereof, and including an upper housing part and a lower housing part connecting with each other, wherein the upper housing part includes a first and a second holes proximate to the opening;

a control panel disposed in an interior of the housing, and including a controlling member secured thereon in response to the first hole and the second hole, and including an engaging head mounted at a front end thereof in response to the opening of the housing to retain a blade;

two buttons fixed in the first and the second holes respectively to correspond to the controlling member of the control panel so as to turn on and off the blade;

wherein a center of the first hole is less than 2.3 cm away from the opening, and the second hole is located at an inner side of the first hole, a center of the second hole is 0.8-1.5 cm away from the center of the first hole.

### BRIEF DESCRIPTION OF THE DRAWINGS

FIG. 1 is a perspective view showing the assembly of an electrosurgical pencil in accordance with a preferred embodiment of the present invention;

FIG. 2 is a perspective view showing the exploded components of the electrosurgical pencil in accordance with the preferred embodiment of the present invention;

FIG. 3 is another perspective view showing the exploded components of the electrosurgical pencil in accordance with the preferred embodiment of the present invention;

FIG. 4 is another perspective view showing the assembly of the electrosurgical pencil in accordance with the preferred embodiment of the present invention;

FIG. 5 is a perspective view showing the exploded components of a conventional electrosurgical pencil.

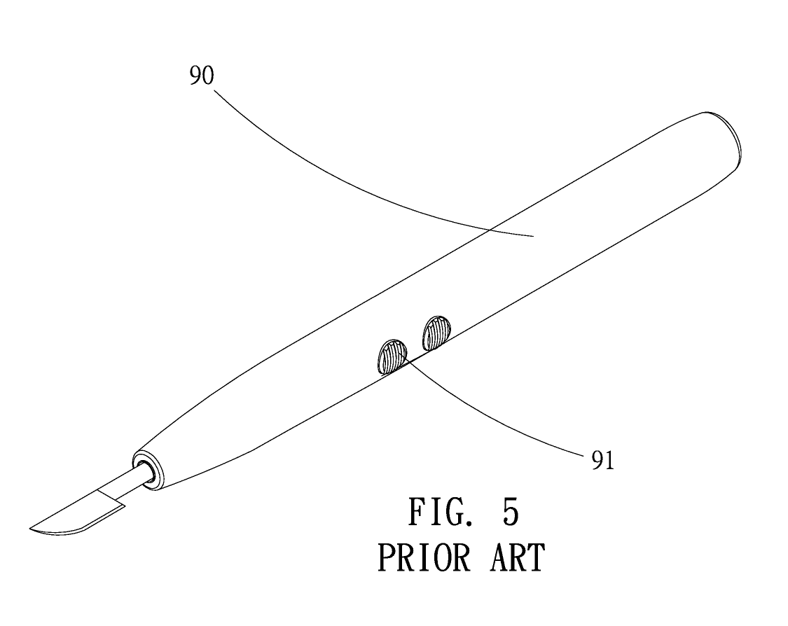

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

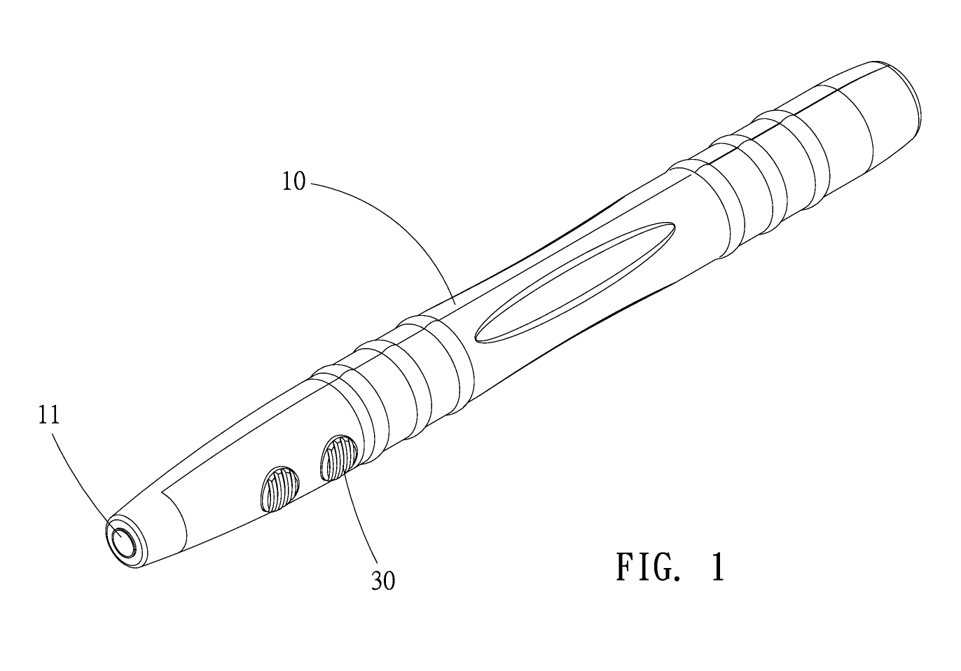

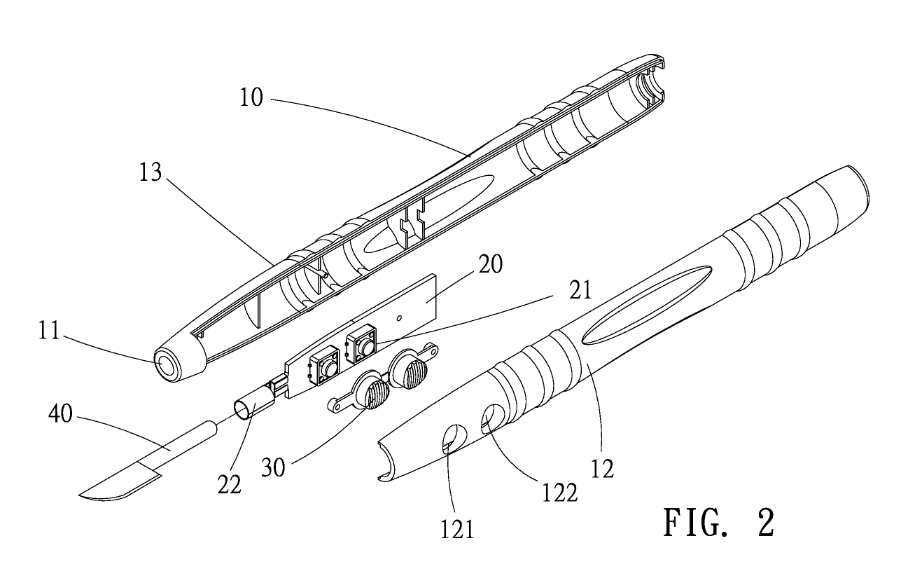

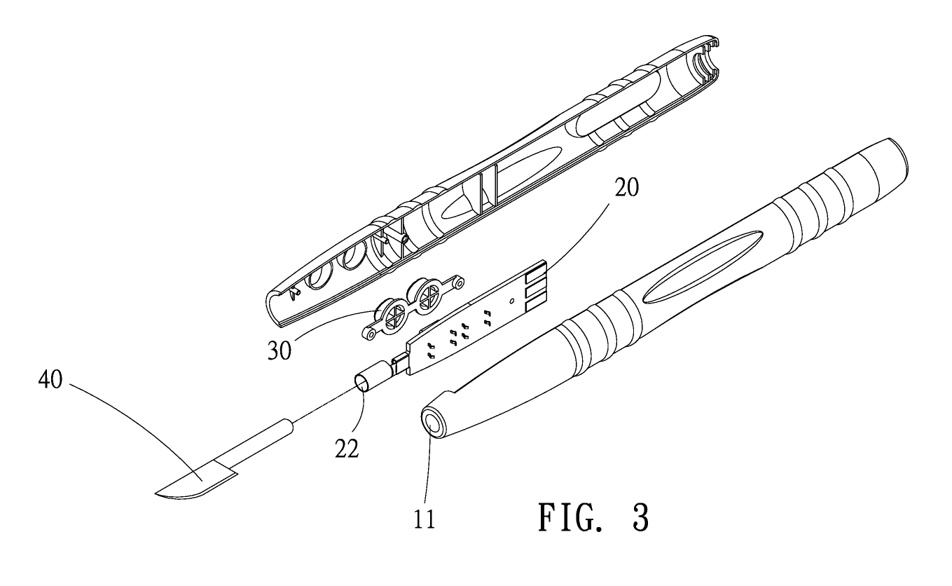

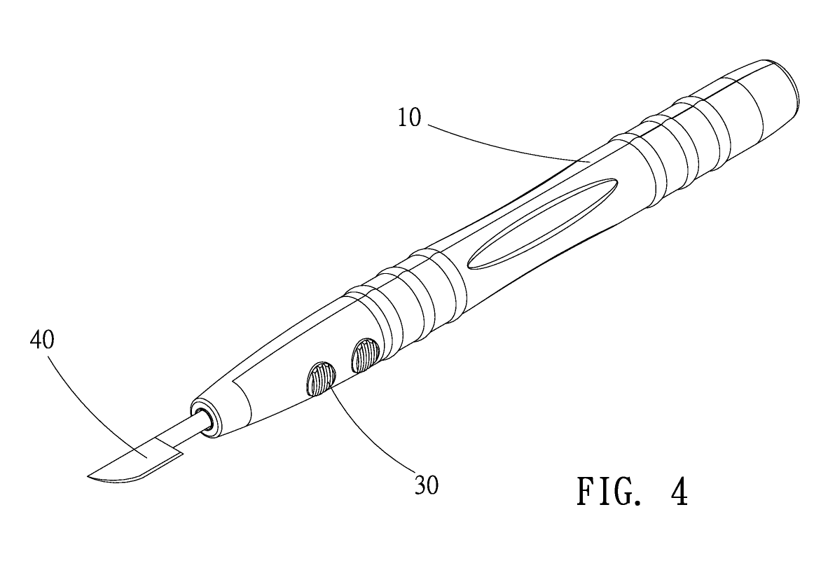

Referring to FIGS. 1-4, an electrosurgical pencil in accordance with a preferred embodiment of the present invention comprises a housing 10, a control panel 20, two buttons 30, and a blade 40.

The housing 10 is an elongated shank member and hollow in an interior thereof, and includes a conical head end with an opening 11 fixed on a front end thereof, and includes an upper housing part 12 and a lower housing part 13 connecting with each other, wherein the upper housing part 12 includes a first and a second holes 121, 122 proximate to the opening 11, and a center of the first hole 121 is less than 2.3 cm away from the opening 11, and the second hole 122 is located at an inner side of the first hole 121, a center of the second hole 122 is 0.8-1.5 cm away from the center of the first hole 121.

The control panel 20 is disposed in an interior of the housing 10, and includes a controlling member 21 secured thereon in response to the first hole 121 and the second hole 122, and includes an engaging head 22 mounted at a front end thereof in response to the opening 11 of the housing 10 to retain the blade 40.

The buttons 30 are fixed in the first and the second holes 121, 122 respectively to correspond to the controlling member 21 of the control panel 20 so as to turn on and off the blade 40.

The characteristic of the present invention is that the two buttons 30 to turn on and off the blade 40 are disposed to proximate to the head end of the electrosurgical pencil, therefore as a user is desired to change an operation of the blade 40 during a surgery, the user does not have to move a forefinger to a middle section of the electrosurgical pencil, thus enabling to switch the blade 40 easily and to obtain a preferred stability.

While we have shown and described various embodiments in accordance with the present invention, it is clear to those skilled in the art that further embodiments may be made without departing from the scope of the present invention.

What is claimed is:

1. An electrosurgical pencil comprising:
a housing being an elongated shank member, and including a conical head end with an opening fixed on a front end thereof, and including an upper housing part and a lower housing part connecting with each other, wherein the upper housing part includes a first and a second holes proximate to the opening;
a control panel disposed in an interior of the housing, and including a controlling member secured thereon in response to the first hole and the second hole, and including an engaging head mounted at a front end thereof in response to the opening of the housing to retain a blade;
two buttons fixed in the first and the second holes respectively to correspond to the controlling member of the control panel so as to turn on and off the blade.

2. The electrosurgical pencil as claimed in claim 1, wherein a center of the first hole is less than 2.3 cm away from the opening, and the second hole is located at an inner side of the first hole, a center of the second hole is 0.8-1.5 cm away from the center of the first hole.
